(19) United States
(12) Patent Application Publication
(10) Pub. No.: US 2001/0053918 A1
(43) Pub. Date: Dec. 20, 2001

(54) Endoscopic piercing instrument

(75) Inventor: Teruo Ouchi, Saitama (JP)

Correspondence Address: GREENBLUM & BERNSTEIN, P.L.C., 1941 ROLAND CLARKE PLACE, RESTON, VA, 20191, US

(73) Assignee: ASHI KOGAKU KOGYO KABUSHIKI KAISHA, Tokyo (JP)

(21) Appl. No.: 09/878,410

(22) Filed: Jun. 12, 2001

(30) Foreign Application Priority Data
Jun. 15, 2000 (JP) P2000-179635

(51) Int. Cl. A61B 17/32

(52) U.S. Cl. 606/170

(57) ABSTRACT

An endoscopic piercing instrument has a needle tube and a flexible sheath. At least those portions of the needle tube and the flexible sheath which are in the neighborhood of their distal ends are each formed of a super-elastic alloy pipe which remains almost straight when left as it is.

### BACKGROUND OF THE INVENTION

The present invention relates to an endoscopic piercing instrument which is inserted through the treatment instrument insertion channel of an endoscope for various purposes such as injecting pharmaceuticals into the affected part of a patient's body.

Endoscopic piercing instruments generally comprise a flexible needle tube with a pointed distal end and a flexible sheath through which the flexible needle tube is passed to be capable of moving back and forth in a longitudinal direction and the needle tube is moved back and forth longitudinally, whereby its distal end is projected beyond or retraced into the flexible sheath.

In the endoscopic piercing instruments, the flexible sheath is typically formed of either a coil pipe made by winding a stainless steel wire in close turns of a specified diameter or a flexible tube made of synthetic resins. The needle tube is typically formed of a stainless steel pipe.

The use of a coil pipe as the flexible sheath has the disadvantage of increasing the thickness of the sheath, which results in an unduly large outside diameter of the piercing instrument. In addition, filth or other foreign body that gets into the space between adjacent turns of the coil is difficult to wash off completely.

As a further problem, the flexible plastic tube and the stainless steel pipe have such a disposition that if they are bent in a small radius of curvature, they undergo plastic deformation and bend permanently. Referring to FIG. 6, if piercing instrument 10 is passed into treatment instrument insertion channel 3 with curved portion 2 in the neighborhood of the tip of endoscope insertion portion 1 being bent in a small radius of curvature, flexible sheath 11 and needle tube 12 that constitute piercing instrument 10 will bend permanently.

As a result, the tip of piercing instrument 10 projecting beyond endoscope insertion portion 1 will not point straightforward to target (affected part of a patient's body) 100 and it often takes considerable time to apply injection in the right position.

The same problem may occur to an endoscope having a treatment instrument erecting table since flexible sheath 11 and needle tube 12 will bend permanently as piercing instrument 10 is passed through the erecting table.

### SUMMARY OF THE INVENTION

An object, therefore, of the present invention is to provide an endoscopic piercing instrument that permits the needle tip to have straightforward access to the target at all times, thereby facilitating piercing in the correct position.

This object of the invention can be attained by an endoscopic piercing instrument comprising a flexible needle tube with a pointed distal end and a flexible sheath through which the flexible needle tube is passed to be capable of moving back and forth in a longitudinal direction, the needle tube being moved back and forth longitudinally, whereby the distal end is projected beyond or retraced into the flexible sheath. In the endoscopic piercing instrument, at least those portions of the needle tube and the flexible sheath which are in the neighborhood of their distal ends are each formed of a super-elastic alloy pipe which remains almost straight when left as it is.

The term “super-elastic” means such a characteristic that if an external stress is applied, the deformation of a large strain region (several percents or several tens percents) exceeding normal elastic strain is caused in accordance with martensitic transformation, and if the external stress is removed, this deformation disappears completely or almost completely in accordance with reverse transformation. The term “super-elastic alloy” means an alloy that exhibits excellent elasticity, like a rubber, under a room temperature.

If the endoscopic piercing instrument has no flexible sheath, an injection socket as an aid for injecting pharmaceuticals and the like may be connected to the basal end of the needle tube having a pointed distal end and at least that portion of the needle tube which is in the neighborhood of its distal end may be formed of the super-elastic alloy pipe.

Preferably, at least that portion of the endoscopic piercing instrument which projects beyond an endoscope into which the endoscopic piercing instrument has been inserted through the treatment instrument insertion channel of the endoscope is formed of the super-elastic alloy pipe.

If desired, the super-elastic alloy pipe may be made of a nickel-titanium base alloy.

The present disclosure relates to the subject matter contained in Japanese patent application No. 2000-179635 (filed on Jun. 15, 2000), which is expressly incorporated herein by reference in its entirety.

### BRIEF DESCRIPTION OF THE DRAWINGS

FIG. 1 shows in partial enlarged section an endoscopic piercing instrument in the first example of the invention as its distal end portion projects from the distal end of an endoscope insertion portion;

FIG. 2 is a side sectional view showing the same endoscopic piercing instrument as its needle tip has been retracted into the flexible sheath;

FIG. 3 is a side sectional view showing the same endoscopic piercing instrument as its needle tip projects from the distal end of the flexible sheath;

FIG. 4 is a sketch showing the actual use of the same endoscopic piercing instrument;

FIG. 5 is a side sectional view of an endoscopic piercing instrument according to the second example of the invention; and

FIG. 6 is a sketch showing the actual use of a related endoscopic piercing instrument.

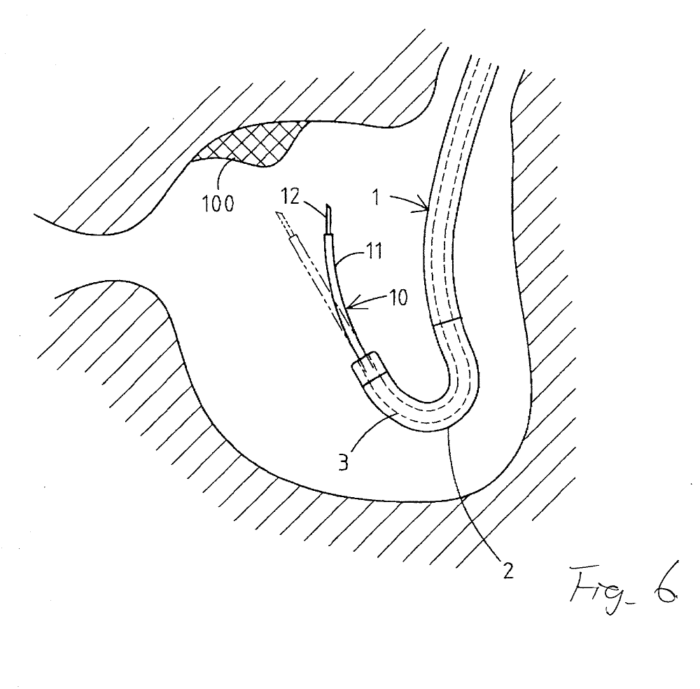

### DESCRIPTION OF THE PREFERRED EMBODIMENT

Several examples of the invention are described below with reference to accompanying drawings.

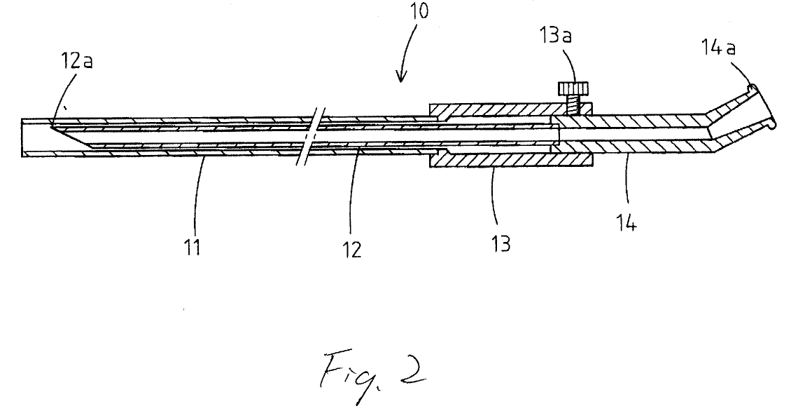

FIG. 2 shows an endoscopic piercing instrument according to a first example of the invention which is generally indicated by 10. It comprises a flexible needle tube 12 and a flexible sheath 11. The flexible needle tube 12 has a sharp pointed tip 12a at the distal end and is passed through the flexible sheath 11 such that it can be moved back and forth in a longitudinal direction. The flexible sheath 11 is to inserted into or removed from the treatment instrument insertion channel of an endoscope.

The flexible sheath 11 and the needle tube 12 are both a pipe that is made of a super-elastic alloy such as a nickel-titanium based alloy and which remains virtually straight if left as it is. Super-elastic alloys as metal have such very high elastic limits that they will not easily bend permanently in spite of their metallic properties such as the ability to withstand compression.

Specifically, the radius of curvature with which the super-elastic alloy pipe can be bent without losing the ability to revert to the initial straight state upon removal of the external force is only about one-fifth to one-eighth the value for the stainless steel pipe.

A grip 13 to be held by the operator between the finger and the thumb is securely coupled to the basal end of the flexible sheath 11. An injection socket 14 with a Luer conical lock socket 14a which can be connected to a syringe cylinder (not shown) or the like is securely coupled to the basal end of the needle tube 12.

The injection socket 14 is fitted into the grip 13 such that it can be moved back and forth in a longitudinal direction. If the operator holds the grip 13 in position and slides the injection socket 14, the needle tube 12 moves back and forth longitudinally through the flexible sheath 11 so that the tip 12a either projects beyond or retracts into the flexible sheath 11.

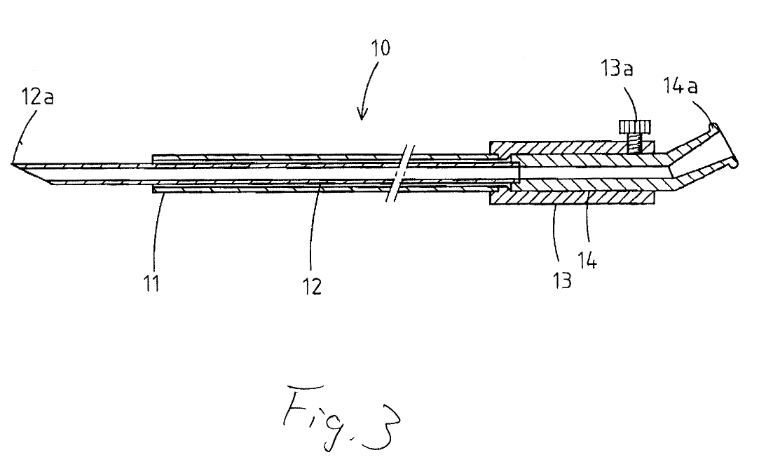

In FIG. 2, the tip 12a of the needle tube 12 is shown to have been retracted into the flexible sheath 11. On the other hand, FIG. 3 shows the needle tube 12 with the tip 12a projecting from the distal end of the flexible sheath 11. A manual screw 13a is fitted on the grip 13. By tightening this manual screw 13a, the injection socket 14 is secured to the grip 13, bringing the needle tube 12 into secure engagement with the flexible sheath 11.

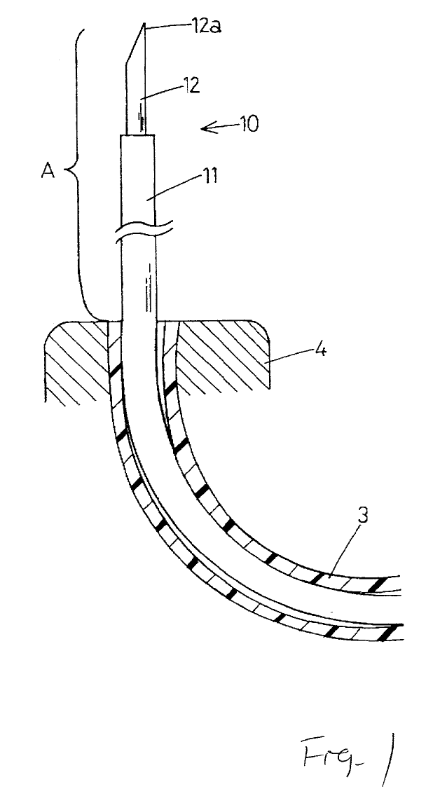

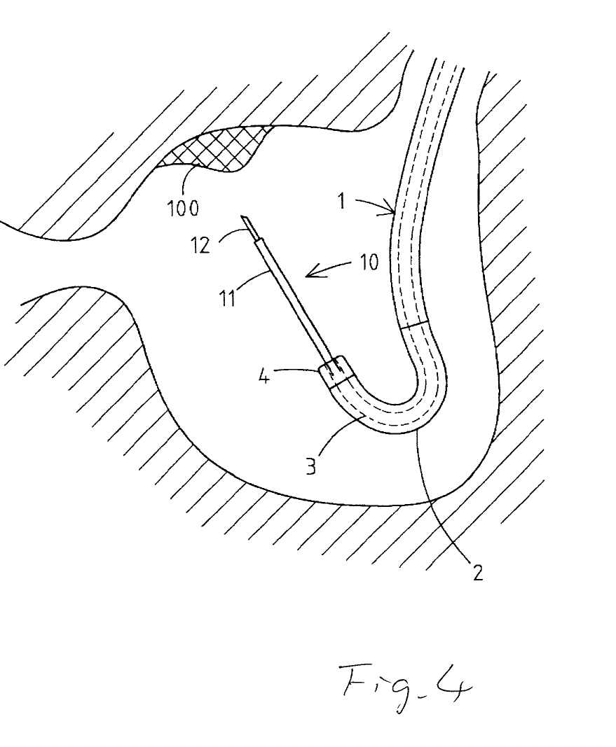

Being constructed in the way described above, the piercing instrument 10 is passed into the treatment instrument insertion channel 3 of an endoscope with the curved portion 2 near the distal end of the endoscope insertion portion 1 being bent with a small radius of curvature as shown in FIG. 4. FIG. 1 is a partial enlarged section of FIG. 4.

The flexible sheath 11 and the needle tube 12 of the piercing instrument 10 protrude from the tip of the distal end body 4 after passing through the treatment insertion channel 3 that has been bent with a small radius of curvature within the curved portion 2 of the endoscope. Since each of the flexible sheath 11 and the needle tube 12 is formed of the super-elastic alloy pipe, the distal end portion of the piercing instrument 10 which projects from the distal end body 4 into the external space will revert to the initial straight state under its own elasticity.

Hence, the piercing instrument 10 projecting from the distal end body 4 follows a straight path to the target area 100 and the operator will find it easy to inject a pharmaceutical or the like toward the right point.

Note that there would be no great adverse effects if that portion of the piercing instrument 10 which does not project from the distal end body 4 at any time of use had some disposition to bend permanently. Therefore, it suffices for the purpose of the invention if at least those portions of the flexible sheath 11 and the needle tube 12 that project from the distal end body 4 and which are indicated by A in FIG. 1 (as guide figures, the projecting portion may be at least 10 mm from the distal end of the flexible sheath 11 and at least 15 mm from the tip 12a of the needle tube 12) are formed of the super-elastic alloy pipe which remains almost straight when left as it is. For most practical purposes, up to about 150 mm of each of the flexible sheath 11 and the needle tube 12 maybe formed of the super-elastic alloy pipe.

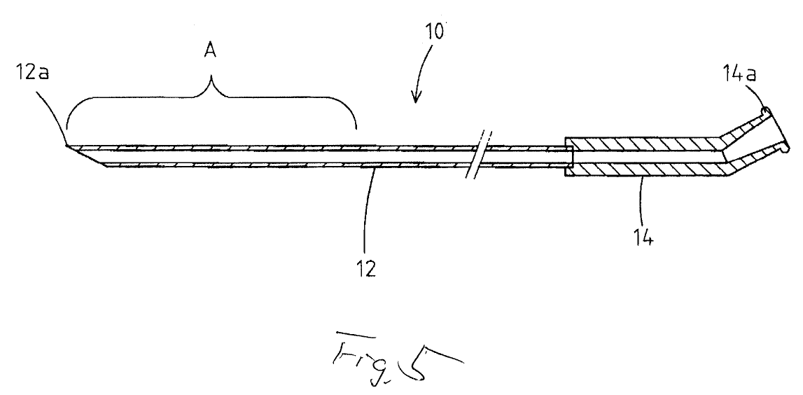

FIG. 5 shows another example of the piercing instrument 10, in which the flexible sheath 11 is absent and the injection socket 14 is simply coupled to the basal end of the needle tube 12. In this case, the needle tube 12 may be formed of the super-elastic alloy pipe and, as in the first example, at least portion A of the piercing instrument 10 which projects from the distal end body 4 of the endoscope need be formed of the super-elastic alloy pipe.

It should also be noted that the applicability of the present invention is by no means limited to endoscopic piercing instruments that are intended for use as an aid to injection of pharmaceuticals or the like and it may be applied to endoscopic piercing instruments intended for other purposes including the collection of tissue samples.

In the endoscopic piercing instrument of the invention, at least those portions of the needle tube and the flexible sheath which are in the neighborhood of their distal ends are each formed of the super-elastic alloy pipe which remains almost straight when left as it is. As a result, the operator of the endoscope can at all times have straightforward access of the needle tip to the target area and finds it easy to pierce the right position.

What is claimed is:

1. An endoscopic piercing instrument comprising a flexible needle tube with a pointed distal end and a flexible sheath through which said flexible needle tube is passed to be capable of moving back and forth in a longitudinal direction, said needle tube being moved back and forth longitudinally, whereby said distal end is projected beyond or retraced into said flexible sheath, wherein at least those portions of said needle tube and said flexible sheath which are in the neighborhood of their distal ends are each formed of a super-elastic alloy pipe which remains almost straight when left as it is.

2. The endoscopic piercing instrument according to claim 1, wherein an injection socket as an aid for injecting pharmaceuticals and the like is connected to the basal end of the needle tube having a pointed distal end and at least that portion of said needle tube which is in the neighborhood of its distal end is formed of the super-elastic alloy pipe.

3. The endoscopic piercing instrument according to claim 1, wherein at least that portion which projects beyond an endoscope into which said endoscopic piercing instrument has been inserted through the treatment instrument insertion channel of said endoscope is formed of the super-elastic alloy pipe.

4. The endoscopic piercing instrument according to claim 1, in which said super-elastic alloy pipe is made of a nickel-titanium base alloy.

5. An endoscopic piercing instrument adapted to be inserted and passed through a treatment instrument insertion channel of an endoscope so that a part of the endoscopic piercing instrument can be protruded from and retracted into a distal end opening of the treatment instrument insertion channel, wherein at least the part of the endoscopic piecing instrument is made of super-elastic alloy, and extends straight when no external force is applied thereto.

6. The endoscopic piercing instrument according to claim 5, comprising:
a flexible needle tube having a sharp pointed tip, wherein a distal end portion of the flexible needle tube, including the sharp pointed tip, constitutes the part of the endoscopic instrument.

7. The endoscopic piercing instrument according to claim 6, wherein the distal end portion of the flexible needle tube has a length of at least 15 mm as measured from the sharp pointed tip.

8. The endoscopic piercing instrument according to claim 6, further comprising:
a flexible sheath through which the flexible needle tube is movably passed, wherein a distal end portion of the flexible sheath constitutes the part of the endoscopic instrument.

9. The endoscopic piercing instrument according to claim 8, wherein the distal end portion of the flexible sheath has a length of at least 10 mm as measured from a distal end opening face of the flexible sheath.

10. The endoscopic piercing instrument according to claim 5, wherein the super-elastic alloy is a nickel-titanium base alloy.
